(19) United States
(12) Patent Application Publication
(10) Pub. No.: US 2020/0365271 A1
(43) Pub. Date: Nov. 19, 2020

(54) METHOD FOR PREDICTING SLEEP APNEA FROM NEURAL NETWORKS

(71) Applicant: FAR EASTERN MEMORIAL HOSPITAL, New Taipe City (TW)

(72) Inventors: TSUNG-WEI HUANG, TAOYUAN CITY (TW); DUAN-YU CHEN, TAOYUAN CITY (TW); SHENG-YE CHEN, TAOYUAN CITY (TW)

(21) Appl. No.: 16/675,494

(22) Filed: Nov. 6, 2019

(30) Foreign Application Priority Data
May 16, 2019 (TW) 108116939

(51) Int. Cl. G16H 50/20; G06N 3/04

(52) U.S. Cl. CPC G16H 50/20; G06N 3/04

(57) ABSTRACT

A method for predicting sleep apnea from neural networks that mainly includes the following steps: a) retrieving an original signal; b) retrieving at least one snoring signal from the original signal by a snoring signal segmentation algorithm and converting the snoring signal into one with one-dimensional vector; c) applying a feature extraction algorithm to process the snoring signal with one-dimensional vector and transform the snoring signal into a feature matrix of two-dimensional vectors; and d) classifying the feature matrix by a neural network algorithm to obtain the number of times of sleep apnea and sleep hypopnea from the snoring signal. The method thereby is able to decide whether the snoring signal has revealed indications of sleep apnea or sleep hypopnea or not.

### BACKGROUND OF THE INVENTION

### 1. Field of the Invention:

The invention relates to a method for predicting sleep apnea that is able to obtain the number of times of sleep apnea and sleep hypopnea from snoring signals of neural networks.

### 2. Description of the Related Art

Both snoring and obstructive sleep apnea (OSA) are resulted from partial airway collapse of the upper airway at sleep. The only difference between snoring and OSA is the severity of the obstruction in the airway. Such problems have influenced at least 4% of population with the effects such as fatigues, tendency to fall asleep, poor memory and depression. Even worse, the problems could cause traffic accidents and illness such as neuropsychiatric disorder, arterial hypertension, cardiovascular disease, stroke and metabolic syndrome. Clinically, polysomnography (PSG) is the main tool for diagnosis of OSA. The PSG requires the patients to sleep at the hospital overnight, including monitoring the sleeping efficiency, number of times of sleep apnea and oxygen saturation. Such inspection process is time-consuming, labor-consuming and expensive. Therefore, it is desired to have a tool for examination and diagnosis of OSA quickly and conveniently and for further monitoring the severity of sleep apnea on a daily basis.

Snoring is the essential symptom of OSA. About 95% of the patients snore during their sleeps. Therefore, self-monitoring of snoring is considered to be a useful tool for them to examine and keep tracking of the condition. On the other hand, snoring analysis has been used to identify pathological respiratory sounds such as wheezes and crackles. However, in the researches, it is difficult to learn the numbers of times of sleep apnea and sleep hypopnea.

Therefore, it's aimed to provide a model for classifying the numbers of times of sleep apnea and sleep hypopnea by neural networks.

### SUMMARY OF THE INVENTION

It is a primary objective of the present invention to provide a method for predicting sleep apnea from neural networks that has a model trained for classifying and concluding the number of times of sleep apnea and sleep hypopnea from snoring signals, so as to further decide whether the snoring signals have revealed indications of sleep apnea or sleep hypopnea or not.

In order to achieve the above objectives, the method for predicting sleep apnea includes the following steps: a) retrieving an original signal; b) applying a snoring signal segmentation algorithm to the original signal to further retrieve at least one snoring signal for segmentation and output a segmented snoring signal vector with one dimension; c) applying a feature extraction algorithm to the snoring signal vector with one dimension to transform the snoring signal vector into a feature matrix of two-dimensional vector; and d) applying a neural network algorithm to the feature matrix of two-dimensional vector for classifying and then providing a result indicating a number of times of sleep apnea and sleep hypopnea within the snoring signal.

Furthermore, the snoring signal segmentation algorithm is performed for segmentation based on a first threshold value and a second threshold value, having a sliding window for linearly inspecting the original signal and calculating a maximum value of the original signal, upon said maximum value being greater than said second threshold value, a snoring signal and a position thereof being recognized, then keeping inspecting the original signal toward a right direction and obtaining a sum value of an absolute value of the snoring signal, upon the sum value being less than the first threshold value, a stop position being set, then keeping inspecting the original signal toward a left direction and obtaining a sum value of an absolute value of the snoring signal, upon the sum value being less than the first threshold value, a start position being set, then the signal fell between said start position and said stop position being segmented and recognized as a snoring signal vector.

The first threshold value is calculated by the following formula:

M=mean(f(Yᵢ>0)),

where M representing the first threshold value, mean representing an average value, f( )representing a down sampling formula and Yᵢ representing a frame vector of the original signal.

The second threshold value is calculated by the following formula:

X=mean(N)+std(N),

where X representing the second threshold value, mean representing an average value, std representing a standard deviation and N representing a natural number which is calculated by the following formula:

N=sort(abs(y̅)),

where sort representing a sorting by numerical order, abs representing an absolute value and y̅ representing the number of vectors the frame vector was segmented into.

In addition, a length of the snoring signal vector is defined to be 25000 frames and the sliding window has window size of 1000.

The feature extraction algorithm has the Mel-Frequency Cepstral Coefficients for the feature extraction process, including procedures of pre-emphasis, framing and windowing, fast Fourier transform, Mel filter bank, nonlinear conversion and discrete cosine transform.

The neural network algorithm is a convolutional neural network algorithm that has a dense convolutional network model as the decision model, and the dense convolutional network model further includes a plurality of dense blocks, a plurality of transition layers and a classification layer. The plurality of transition layers further includes a convolution process and a pooling process and the classification layer is a softmax layer. And the plurality of dense blocks further includes a dense layer, a batch normalization-rectified linear units-convolution layer and a growth rate.

With structures disclosed above, the present invention has the snoring signal segmentation algorithm, the feature extraction algorithm and the neural network algorithm integrated to efficiently process the original signal and to further obtain the number of times of sleep apnea and sleep hypopnea from the snoring signal retrieved from the original signal, so as to decide whether the snoring signal has revealed indications of sleep apnea or sleep hypopnea or not. Such method has overcome the shortcomings of inability to predict or obtain an indication of sleep apnea and sleep hypopnea.

### BRIEF DESCRIPTION OF THE DRAWINGS

FIG. 1 is a flow diagram of the present invention;

FIG. 2 is a schematic diagram illustrating an operation process of the present invention;

FIG. 3A is a schematic diagram of an original signal according to the present invention;

FIG. 3B is a schematic diagram of the original signal after normalization according to the present invention;

FIG. 3C is a schematic diagram of partial of the original signal according to the present invention;

FIG. 3D is a schematic diagram of the partial original signal after normalization being inspected for recognizing snoring signals according to the present invention;

FIG. 3E is a schematic diagram of a snoring signal being segmented according to the present invention;

FIG. 3F is a schematic diagram of a first single snoring signal after segmentation according to the present invention;

FIG. 3G is a schematic diagram of a second single snoring signal after segmentation according to the present invention;

FIG. 3H is a schematic diagram of a third single snoring signal after segmentation according to the present invention;

FIG. 3I is a schematic diagram of a fourth single snoring signal after segmentation according to the present invention;

FIG. 3J is a schematic diagram of a fifth single snoring signal after segmentation according to the present invention;

FIG. 3K is a schematic diagram of a sixth single snoring signal after segmentation according to the present invention;

FIG. 3L is a schematic diagram of a seventh single snoring signal after segmentation according to the present invention;

FIG. 4 is a schematic diagram illustrating procedures of the Mel-Frequency Cepstral Coefficients for a feature extraction process according to the present invention;

FIG. 5 is a schematic diagram illustrating a dense convolutional network model according to the present invention;

FIG. 6 is a schematic diagram illustrating structure of a dense block according to the present invention;

FIG. 7A is a schematic diagram illustrating a decision model of sleep apnea according to the present invention; and

FIG. 7B is a schematic diagram illustrating a prediction process of the decision model of sleep apnea according to the present invention.

### DETAILED DESCRIPTION OF THE PREFERRED EMBODIMENT

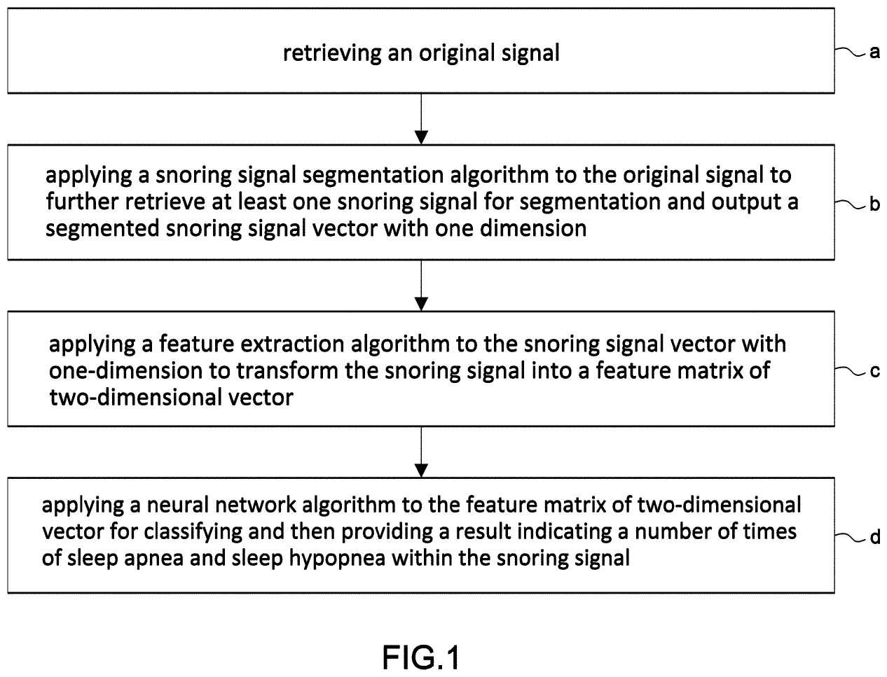

Referring to FIGS. 1-7B, the method for predicting sleep apnea from neural network includes the following steps.

Step a: retrieving an original signal Y. In this embodiment, polysomnography (PSG) has been performed on multiple subjects. The variables include the apnea-hypopnea index (AHI), snoring index and minimum oxygen saturation (MOS). The AHI is the number of times obstructive apnea and hypopnea happened per hour of sleep. Apnea is defined when the inhalation and exhalation stops for at least 10 seconds and hypopnea is defined when the baseline ventilator value is decreased by 50% or more and the oxygen saturation is decreased by 4% or more, and such reduction lasts more than 10 seconds. When performing PSG, the sound of snoring is recorded by a mini-microphone placed on a position above the suprasternal notch. But the present invention is not limited to such application.

Step b: applying a snoring signal segmentation algorithm G₁ to the original signal Y to further retrieve at least one snoring signal B for segmentation and output the segmented snoring signals with one-dimensional vector S. Since the original signal Y is the audio file recorded all night, the data has to be processed in advance. The snoring signal segmentation algorithm G₁ is applied to automatically sort out and segment the snoring signals from the original signal Y. But the present invention is not limited to such application.

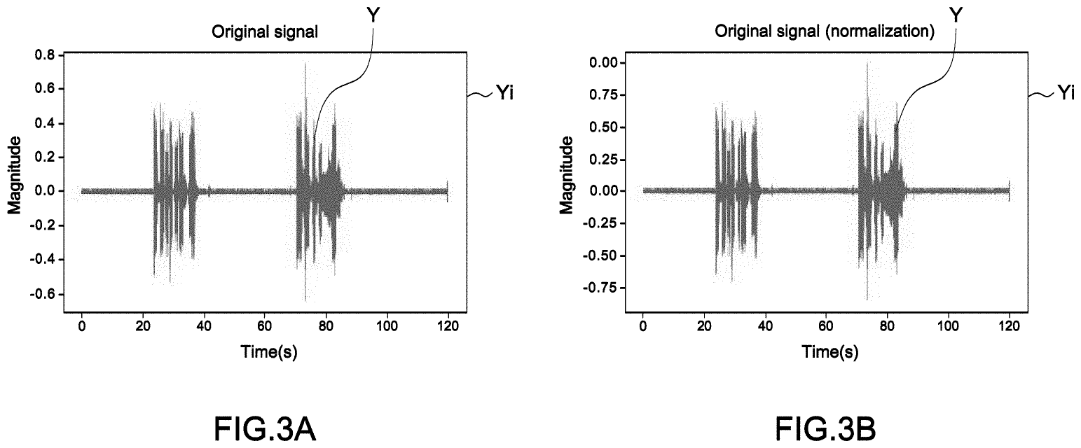

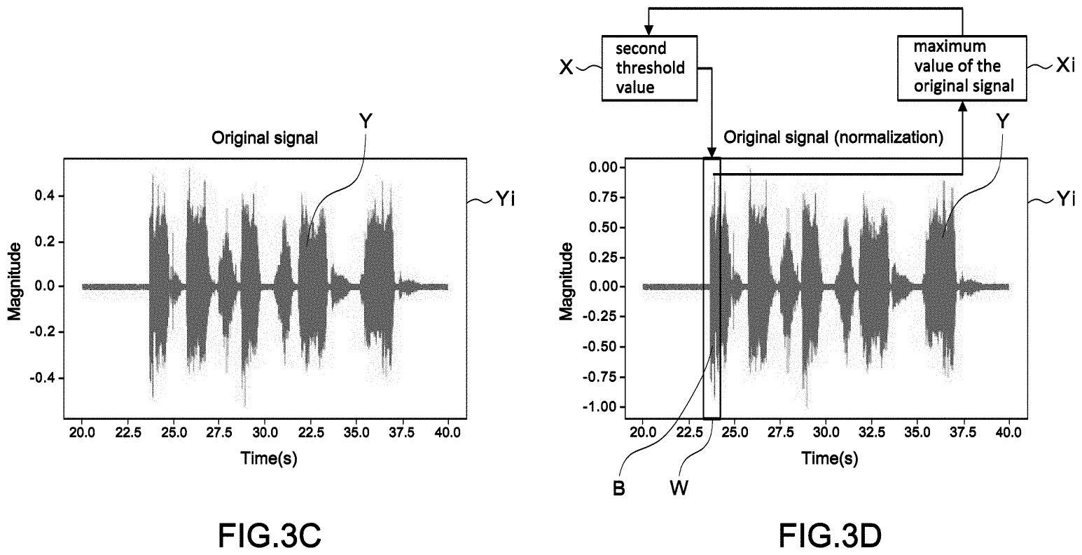

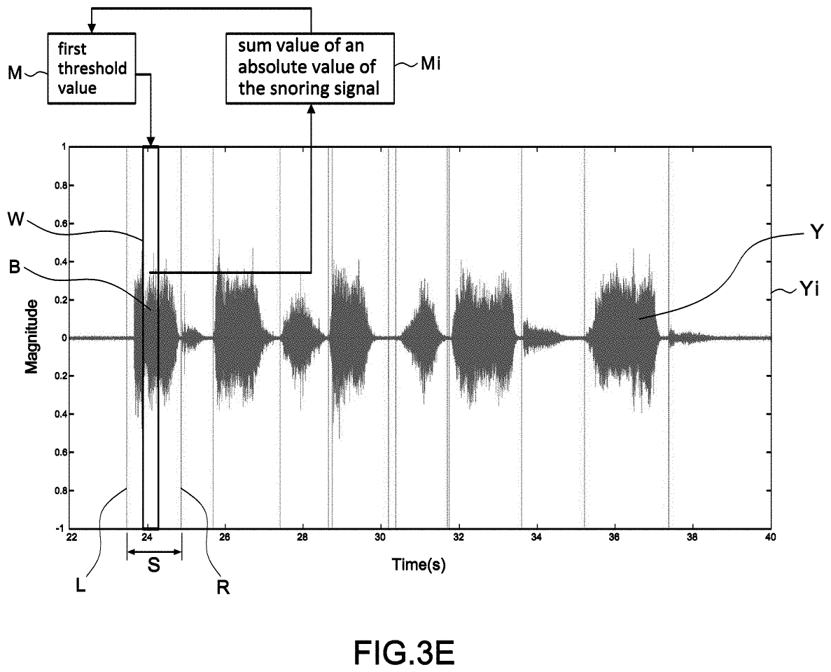

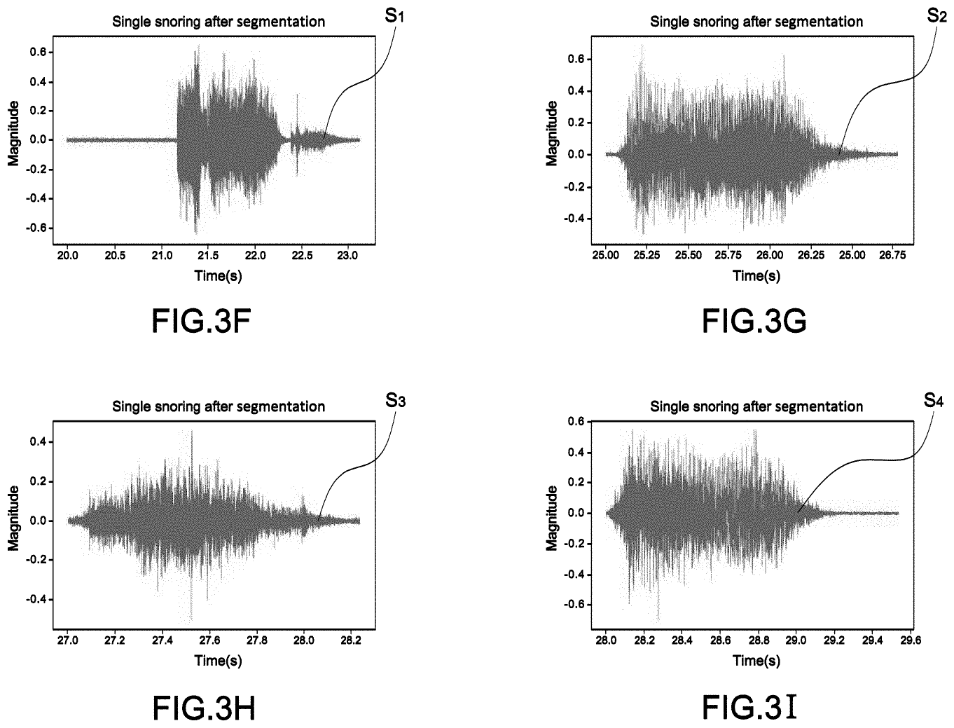

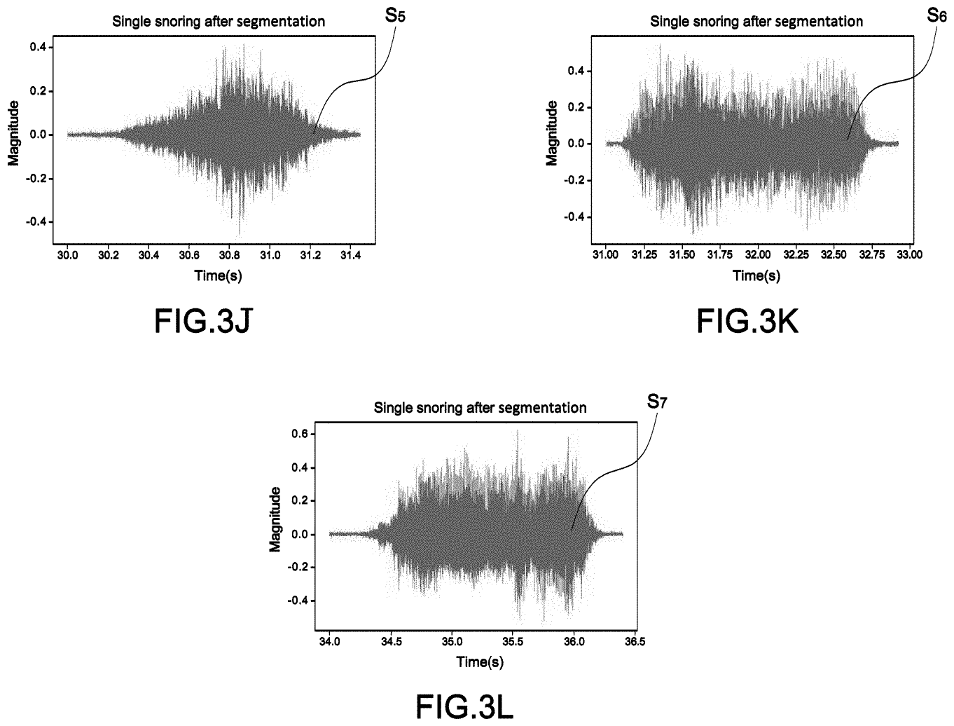

With reference to FIGS. 3A-3L, the longitudinal direction is the magnitude and the transverse direction is the times. In this embodiment, the snoring signal segmentation algorithm G₁ is performed for segmentation based on a first threshold value M and a second threshold value X. The snoring signal segmentation algorithm G₁ has a sliding window W for linearly inspecting the original signal Y and, as illustrated in FIG. 3D, the algorithm G₁ calculates a maximum value Xi of the original signal Y during the inspection. When the maximum value Xi is greater than the second threshold value X, a snoring signal B and a position of the snoring signal B are recognized. Then the inspection continues toward a right direction along the sliding window W and, as illustrated in FIG. 3E, a sum value Mi of an absolute value of the snoring signal Y is further obtained. When the sum value Mi is less than the first threshold value M, a stop position R is set. Then the inspection continues toward a left direction and a sum value Mi of an absolute value of the snoring signal Y is obtained again. When the sum value Mi is less than the first threshold value M, a start position L is set.

Then the signal fell between the start position L and the stop position R is segmented and recognized as a snoring signal vector S with one dimension as illustrated in FIGS. 3F-3L. After the segmentation, a first single snoring signal vector S₁, a second single snoring signal vector S₂, a third single snoring signal vector S₃, a fourth single snoring signal vector S₄, a fifth single snoring signal vector S₅, a sixth single snoring signal vector S₆ and a seventh single snoring signal vector S₇ are recognized. Since the snoring signal vectors are required to have the same length for further processing, a length of 25000 frames is set in this embodiment.

In addition, the formula for calculation of the first threshold value M is

M=mean(f(Yᵢ>0)),

where M represents the first threshold value M; mean represents an average value; f( )represents a downsampling formula; Yᵢ represents a frame vector Yi of the original signal Y every 2 minutes downsampled to a dimension of 400. The downsampling process has the frame vectors Yi equally segmented to the same dimension and retrieves a maximum value of each segment. The frame vectors Yi are downsampled to a vector of 1*400, thereby producing a more reliable value of the first threshold value M.

The formula for calculation of the second threshold value X is

X=mean(N)+std(N),

where X represents the second threshold value X; mean represents an average value; std represents a standard deviation; N represents a natural number calculated by a formula of

N=sort(abs(y̅)),

where sort represents a sorting by numerical order; abs represents an absolute value; y̅ representing the number of vectors the frame vector was segmented into. In other words, the number of vectors is the result of the length of the frame vector Yi dividing the size of the sliding window W. When the sliding window W has window size of 1000, the natural number is obtained and the second threshold value X can be further obtained.

Step c: applying a feature extraction algorithm G₂ to the snoring signal vector S with one dimension to transform the snoring signal vector S into a feature matrix A of two-dimensional vector. Thereby the original signal Y is segmented into the plurality of single snoring signals S₁, S₂, S₃, S₄, S₅, S₆, S₇ for further processing of the feature extraction algorithm G₂. The feature extraction algorithm G₂ has the Mel-Frequency Cepstral Coefficients

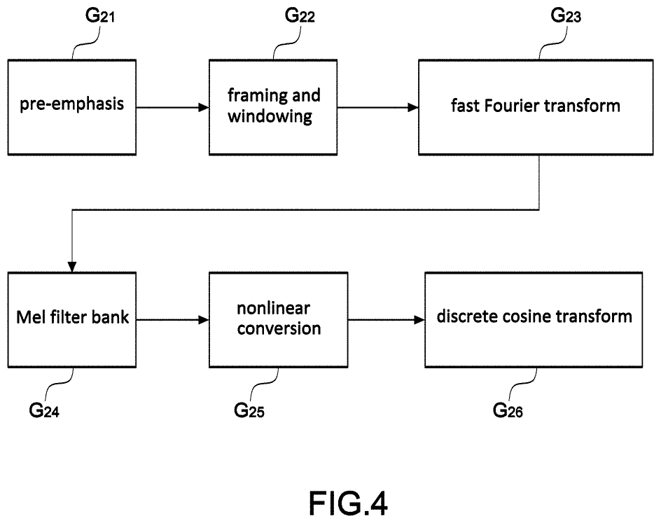

(MFCC) for the feature extraction process, including procedures of pre-emphasis G₂₁, framing and windowing G₂₂, fast Fourier transform G₂₃, Mel filter bank G₂₄, nonlinear conversion G₂₅ and discrete cosine transform G₂₆ as illustrated in FIG. 4.

The pre-emphasis G₂₁ aims to compensate for the attenuated portion of the single snoring signals S₁, S₂, S₃, S₄, S₅, S₆, S₇ by a process defined as:

Hₚᵣₑₑₘ(z)=1−αₚᵣₑₑₘz⁻¹,

where Hₚᵣₑₑₘ represents the result after the pre-emphasis process G₂₁ and αₚᵣₑₑₘ represents the input signal of sounds.

The framing and windowing G₂₂ has the single snoring signals S₁, S₂, S₃, S₄, S₅, S₆, S₇ divided into shorter frames, each of which has a length of 20-40 milliseconds. In order to avoid significant changes between two frames, there is an overlapping area of 10 milliseconds between each frames and each frame is multiplied by the Hamming window to enhance the continuity between the borders of the frames. The signals close to the borders of the frames are slowly faded out to avoid the discontinuity and the energy spectrum of noise would be weakened, thereby the peak of the sine wave of the signals would be relatively prominent as well. If there is obvious discontinuity between each frames, there will be other misguiding energy distribution in the next fast Fourier transform process, causing misjudgment of the analysis in the process. Therefore, the signals have to be multiplied by the Hamming window during this step.

The fast Fourier transform G₂₃ is applied to convert the signals from the time domain to the frequency domain, and fast Fourier transform is the fast algorithm of discrete Fourier transform.

The Mel filter bank G₂₄ is a band-pass filter that overlaps with each other. Based on the Mel scale, it is linear under the frequency of 1 Hz and logarithmic thereon. The Mel scaling process is defined as:

$$mel = 2595 {log}_{10} ( 1 + \frac{f}{700} ) ,$$

where mel represents the result of the Mel filter bank; f represents the input of the filter bank; and the numbers 2595 and 700 are fixed numbers that have been widely used in the filter process in many researches. The energy spectrum is multiplied by a set of 16 triangular band-pass filters and thus we use the Mel Frequency as the spectrum of the 16 filters.

The discrete cosine transform G₂₆ is applied for calculation of the MFCCs in each frame, and the conversion is based on the following equation:

Σₖ₌₁^{N}log(Y(i))* cos[mx(k−0.5)xπ÷N]

Thereby the snoring signal B can be converted into the MFCCs feature matrix A.

Step d: applying a neural network algorithm G₃ to the feature matrix A of two-dimensional vector for classifying and then providing a result indicating a number of times of sleep apnea and sleep hypopnea within the snoring signal B. After the feature extraction process, the snoring signal B is converted into a two-dimensional vector, and, as most image classification process are performed by the neural network algorithm G₃, we can also apply the neural network algorithm G₃ for classifying the feature matrix A. But the present invention is not limited to such application.

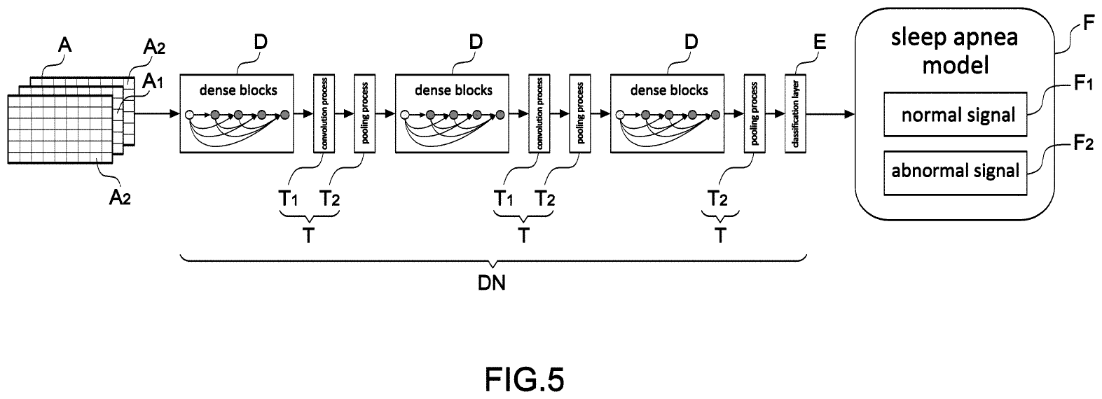

In this embodiment, the neural network algorithm G₃ is a convolutional neural network algorithm which has a dense convolutional network (DN) model as a decision model. As illustrated in FIG. 5, the dense convolutional network model includes a plurality of dense blocks D, a plurality of transition layers T and a classification layer E. The transition layers T includes a convolution process T₁ and a pooling process T₂, and the classification layer E is a softmax layer.

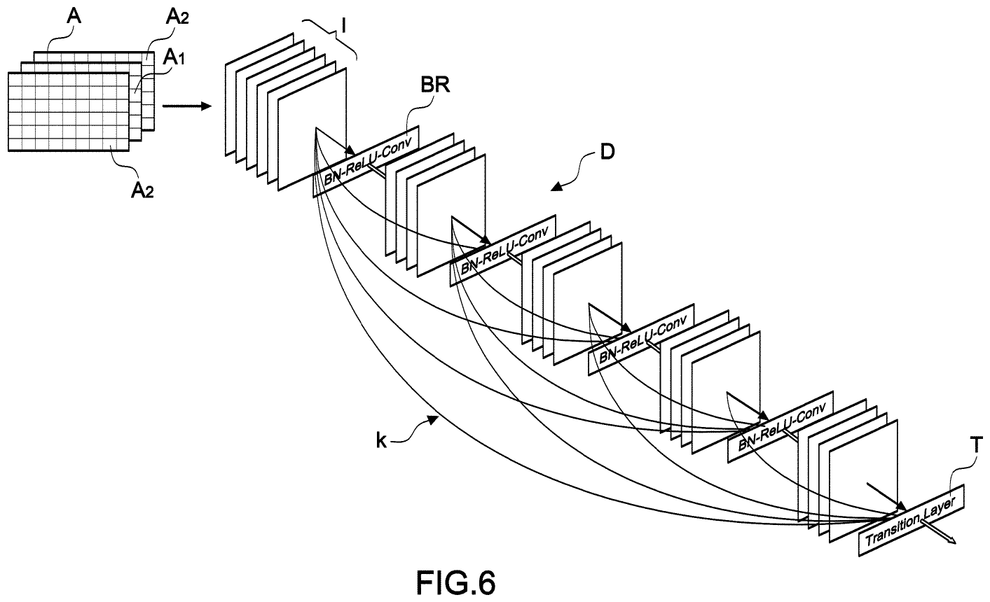

Further referring to FIG. 6, the dense blocks D includes a dense layer I, a batch normalization-rectified linear units-convolution layer BR and a growth rate k. The growth rate k is the number of feature maps output from each layer. Since the DN model consists of multiple connected dense blocks D and transition layers T, and is finally connected to the classification layer E, the dense blocks D are densely connected convolutional neural networks. The snoring signal B is segmented and labeled for further training. For instance, if the feature matrix A does not contain signals of sleep apnea and sleep hypopnea, it is labeled normal A₁; if the feature matrix A contains signals of sleep apnea or sleep hypopnea, it is labeled abnormal A₂. After sending the labeled signals into the DN model, a sleep apnea model F is produced and ready for operation. However, the present invention is not limited to such application.

Within the dense blocks D, any two layers are directly connected; therefore, the input of each layer in the network is the output of its previous layer, and the feature map of each layer is also transmitted directly to all the descendent layers. Such approach employs the DN model to make efficient use of all-level features. The transition layers T are designed to reduce the size of the feature matrix. Since the final layer of the output from the dense blocks D, the model can be very large. Therefore, the transition layers T are employed to reduce the amount of the parameters greatly. With such structures, the DN model solves the problem of gradient vanishing occurred when the network architecture is too deep and has the resistance to over-fitting.

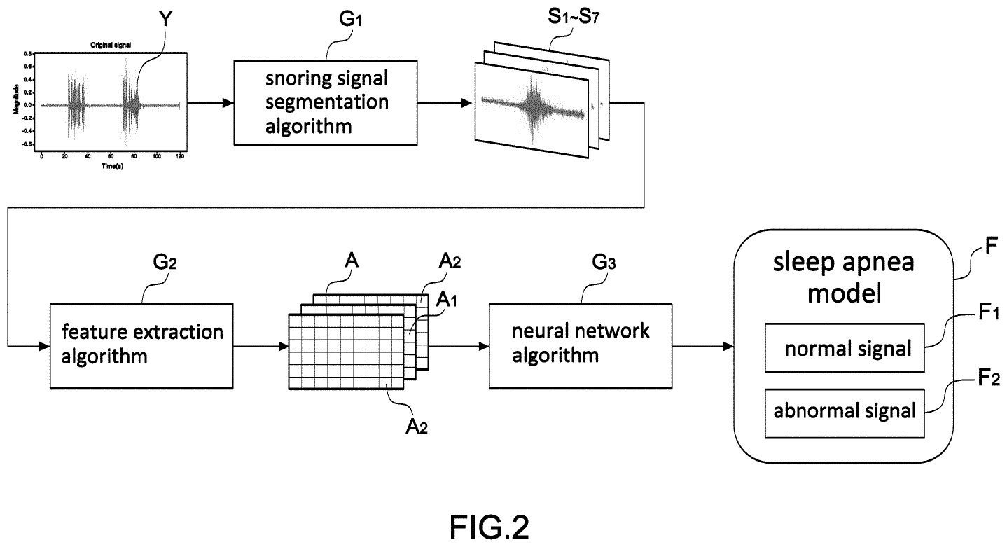

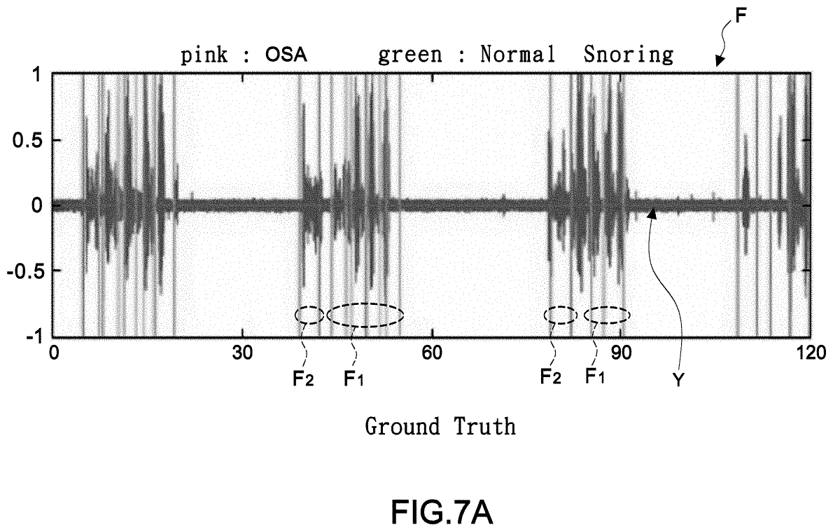

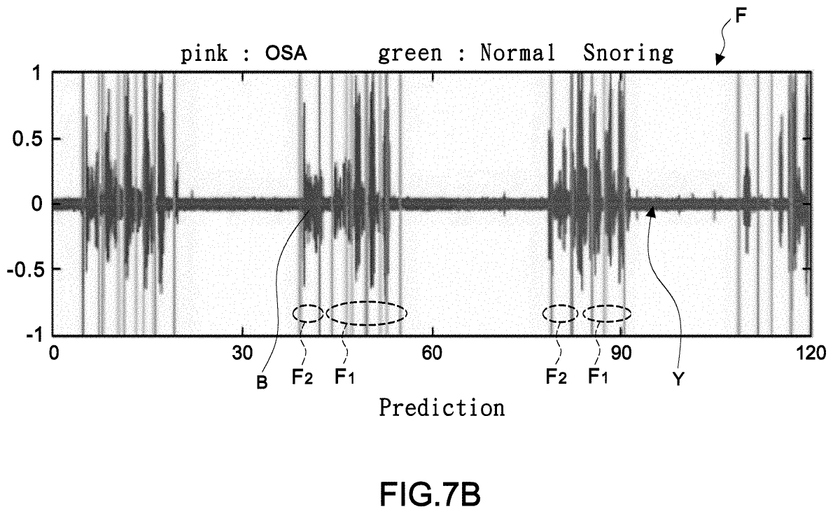

Further referring to FIG. 2, the sleep apnea model F is able to predict a normal signal F₁ and an abnormal signal F₂. And as illustrated in FIG. 7A, the sleep apnea model F has a ground truth displayed in blue color, the normal signal F₁ as normal snoring displayed in green color and the abnormal signal F₂ as obstructive sleep apnea (OSA) displayed in pink color for establishing a ground data. Then referring to FIG. 7B, the snoring signal B is inserted into the sleep apnea model F displayed in red color for deciding whether the snoring signal B is a normal signal F₁ or an abnormal signal F₂ and further predicting whether it is sleep apnea or not.

What is claimed is:

1. A method for predicting sleep apnea from neural networks, comprising steps as following:
retrieving an original signal;
applying a snoring signal segmentation algorithm to said original signal to further retrieve at least one snoring signal for segmentation and output a segmented snoring signal vector with one dimension;
applying a feature extraction algorithm to said snoring signal vector with one-dimension to transform the snoring signal into a feature matrix of two-dimensional vector; and
applying a neural network algorithm to said feature matrix of two-dimensional vector for classifying and then providing a result indicating a number of times of sleep apnea and sleep hypopnea within the snoring signal.

2. The method for predicting sleep apnea from neural networks as claimed in claim 1, wherein the snoring signal segmentation algorithm is performed for segmentation based on a first threshold value and a second threshold value, having a sliding window for linearly inspecting the original signal and calculating a maximum value of the original signal, upon said maximum value being greater than said second threshold value, a snoring signal and a position thereof being recognized, then keeping inspecting the original signal toward a right direction and obtaining a sum value of an absolute value of the snoring signal, upon the sum value being less than the first threshold value, a stop position being set, then keeping inspecting the original signal toward a left direction and obtaining a sum value of an absolute value of the snoring signal, upon the sum value being less than the first threshold value, a start position being set, then the signal fell between said start position and said stop position being segmented and recognized as a snoring signal vector with one dimension.

3. The method for predicting sleep apnea from neural networks as claimed in claim 2, wherein a formula for calculation of the first threshold value is M=mean(f(Yᵢ>0)), where M representing the first threshold value, mean representing an average value, f( )representing a down sampling formula and Yᵢ representing a frame vector of the original signal, and a formula for calculation of the second threshold value is X=mean(N)+std(N), where X representing the second threshold value, mean representing an average value, std representing a standard deviation and N representing a natural number calculated by a formula: N=sort(abs(y̅)), where sort representing a sorting by numerical order, abs representing an absolute value and y̅ representing the number of vectors the frame vector was segmented into.

4. The method for predicting sleep apnea from neural networks as claimed in claim 2, wherein a length of the snoring signal vector is defined to be 25000 frames.

5. The method for predicting sleep apnea from neural networks as claimed in claim 2, wherein the sliding window has window size of 1000.

6. The method for predicting sleep apnea from neural networks as claimed in claim 1, wherein the feature extraction algorithm has the Mel-Frequency Cepstral Coefficients for the feature extraction process, including procedures of pre-emphasis, framing and windowing, fast Fourier transform, Mel filter bank, nonlinear conversion and discrete cosine transform.

7. The method for predicting sleep apnea from neural networks as claimed in claim 1, wherein the neural network algorithm is a convolutional neural network algorithm, having a dense convolutional network model as a decision model.

8. The method for predicting sleep apnea from neural networks as claimed in claim 7, wherein the dense convolutional network model includes a plurality of dense blocks, a plurality of transition layers and a classification layer.

9. The method for predicting sleep apnea from neural networks as claimed in claim 8, wherein the plurality of transition layers includes a convolution process and a pooling process, and the classification layer is a softmax layer.

10. The method for predicting sleep apnea from neural networks as claimed in claim 8, wherein the plurality of dense blocks includes a dense layer, a batch normalization-rectified linear units-convolution layer and a growth rate.
